(19) United States
(12) Patent Application Publication
(10) Pub. No.: US 2007/0255074 A1
(43) Pub. Date: Nov. 1, 2007

(54) Method for Exchanging Anions of Tetraalkylammonium Salts

(76) Inventors: Joe D. Sauer, Baton Rouge, LA (US); Christopher S. Knight, Prairieville, LA (US); Charles R. Everly, Baton Rouge, LA (US)

Correspondence Address: Edgar E. Spielman;Albemarle Corporation, 451 Florida Street, Baton Rouge, LA, 70801, US

(21) Appl. No.: 11/547,338

(22) PCT Filed: Mar. 25, 2005
(86) PCT No.: PCT/US05/10168

§ 371 Date: Feb. 9, 2007

Related U.S. Application Data
(60) Provisional application No. 60/557,106, filed on Mar. 26, 2004.

(51) Int. Cl. C07C 211/63; C07C 211/62

(52) U.S. Cl. 564/281

(57) ABSTRACT

A method of converting a tetraalkylammonium salt of a first anion to a tetraalkylammonium salt of a second anion is described which involves forming a solution comprising the tetraalkylammonium salt of the first anion in a solvent which includes water and a polar organic co-solvent, contacting the solution with an ion exchange resin in the form of the second anion and forming a tetraalkylammonium salt of the second anion in solution and an ion exchange resin complexed with the first anion. The solution containing the tetraalkylammonium salt of the second anion can be recovered.

### CROSS REFERENCE TO RELATED PATENTS AND PATENT APPLICATIONS

The present application is a non-provisional of, and claims the benefit of, copending U.S. Provisional Patent Application Ser. No. 60/557,106, filed Mar. 26, 2004, which is relied on herein and incorporated herein by reference in its entirety. The subject matter of the present invention is related to a copending and commonly assigned U.S. Patent Application having the title “Methods for the synthesis of quaternary ammonium compounds and compositions thereof”, having attorney docket number 23138/09008, which was filed on the same date as the present application.

### BACKGROUND OF THE INVENTION

(1) Field of the Invention

The present invention relates to a method of exchanging the anion of a quaternary alkylammonium salt with a different anion, and more particularly to a method of exchanging the anion of a quaternary tetraalkylammonium salt with a different anion by using an ion exchange resin.

(2) Description of the Related Art

The two most widespread alternative wood preservatives are quaternary ammonium compounds (quats) and copper azoles. Because of the marketplace trend away from metal-containing wood preservatives, there has been increased interest in the use of quats as wood preservatives, as biocides, and as surfactants in many different applications. Uses of quat compounds are described generally in U.S. Pat. Nos. 3,301,815, 3,366,672, 4,365,030, and 4,444,790, among others.

Quats are a diverse group of compounds that find utility not only in the wood preservative/biocide industry, but also in such industries as hair care products, cleaning products, fabric softeners, pharmaceuticals, surfactants, deodorants, mouthwashes, preservatives, emulsifiers, cosmetics, and ore mining. Quats are loosely defined as a group of compounds in which a nitrogen atom is joined to four organic radicals. Due to the net positive charge on the nitrogen atom in the quaternary ammonium compound, quats are always found associated with one or more anions. The associated anions are termed counterions, and the combination of the quaternary ammonium compound and the anion is termed a quat salt.

Commercial classes of quat compounds have historically developed in a fairly orderly fashion. The general compound alkyltrimethyl ammonium chloride (ATMAC) is representative of class I type quats. These quats are composed of a nitrogen having substituent groups comprising three methyl groups and one alkyl group. Representative of class II quat compounds is the general compound alkyldimethylbenzyl ammonium chloride (ADBAC)—a nitrogen having two substituent methyl groups and in addition, a substituent alkyl group and a substituent alkyl aryl group. The general compound alkyldimethyl(ethylbenzyl)ammonium chloride (ADEBAC) is representative of class III quat compounds. These compounds comprise a nitrogen having substituent groups of two methyl groups, one alkyl group and another alkylaryl group in which the alkyl is other than methyl. Finally, the general compound dialkyldimethyl ammonium chloride (DADMAC) is representative of class IV quat compounds. The class IV quats typically comprise a nitrogen having two methyl group substituents and two alkyl group substituents. One type of class IV quats have substituent alkyl groups of different chain lengths, and can be termed “double-tail” quats. A special class of double-tail quats includes those in which the alkyl substituent groups are of the same chain length. These quats can be referred to as “twin-tail” quats.

Moving from a lower class to a higher class of quats generally provides the consumer with compounds that have more efficacious surface activity and/or biologically activity. Often, the higher classes of quats will be more durable or resistant to deactivation in actual applications. As one might expect, moving from a lower class quat to a higher class quat commonly results in higher active costs—at least on a per pound basis.

Higher classes of surface active quats present special challenges for the processor and the formulator. The solubility of quats of higher classes can be substantially altered from the lower class counterparts, often with the more complicated species being less soluble in water. However, solubility depends to some degree upon the identity of the quat counterion, and solubility can be modified by alteration of the counter-ion (i.e., anion). In many cases, use of “larger” anions can bring the total surface active structure more into balance (e.g., soft acid:soft base combinations). This generally results in a quat salt that displays improved solubility and stability when in aqueous solution.

Methods for the production of quaternary ammonium compounds are generally known in the art. Basic chemistry texts teach that the end-product of the reaction of ammonia or of an amine with an alkyl halide is a quaternary ammonium salt. See, e.g., Noller, C. R., Textbook of Organic Chemistry, 2^{nd} Ed., p. 188 et seq., W. B. Saunders Co., Philadelphia (1961), and March, J., Advanced Organic Chemistry, 3^{rd} Ed., pp. 364, 365, John Wiley & Sons, New York (1985). Numerous other publications, for example U.S. Pat. Nos. 5,308,363, 5,438,034, 5,523,487, 5,855,817, 6,784,317, 6,090,855, 6,485,790, and U.S. Patent Application Publication US 2003/0023108, report specific methods of producing quats having particular types of substituent groups and particular counter-anions.

U.S. Pat. No. 2,295,504 to Shelton, for example describes the formation of cetyltriethylammonium salts by reacting triethylamine with cetyl iodide, and states that corresponding stearyl, myristyl and oleyl triethyl ammonium iodides can be made in a similar manner. Similar reactions using alkyl bromides and alkyl chlorides are also reported. The reaction of a quat bromide with silver sulfate to form the quat sulfate and insoluble silver bromide is described. More complex quats are formed by sequential reactions with different alkyl halides, each adding a particular alkyl group to the nitrogen.

De Benneville, in U.S. Pat. No. 2,994,699, reports the formation of ketonic quaternary ammonium compounds and states that while it is advantageous to initially prepare the compounds in the form of halides, different anions can be supplied by either metathesis or ion exchange. Examples include conversion of the halide salt to the hydroxyl salt by reaction of the quat halide with silver oxide, or the like, and the subsequent conversion of the hydroxyl to any desired anion form by acidifying with an acid of the desired anion. The patent states that any of the quat salts described therein can be converted to any other anion form by contacting it with an anion-exchange resin in the desired anion form. No examples of an ion exchange anion transfer are provided.

In U.S. Pat. No. 3,190,919, Swanson describes two methods of exchanging anions of quat salts. The first method is described as repeatedly contacting an organic solution of the quat with an aqueous solution containing the desired anion. However, the method is described as being cumbersome to carry out commercially, and to be expensive and somewhat hazardous, due to the large amounts of solvents employed. The use of ion exchange resins is described as the second method for exchanging quat anions. Here, the quat is passed through a column of ion exchange resin in the form of the final desired quat anion. When it is said that an ion exchange resin is in the form of a particular anion, it is meant that the resin is complexed with that anion. The resin releases the desired anion and absorbs the starting anion. Swanson describes the method as being useful on a laboratory scale but highly impractical on a commercial scale, and states that a dilute solution of the quat must be used, requiring large amounts of solvents. In addition, he states that ion exchange resins are expensive and must be periodically replaced. Large quantities of resins must be employed, first because of low capacity inherent in exchange resins and second because of low transfer coefficients attendant with the use of non-protic solvents, such as hydrocarbons. Further, he states, regeneration is very difficult when non-protic solvents are employed. He concludes that the use of an ion exchange resin for exchange of quat anions is highly uneconomical.

To overcome these problems, Swanson describes a process wherein a quat salt having an anion of a volatile acid, such as sulfurous, is dissolved in a non-water soluble organic solvent, such as kerosene, and the organic solution is contacted with an aqueous solution of a non-volatile acid, such as sulfuric. An inert gas—air, for example—is purged through the mixture, stripping out the volatile acid and leaving the quat salt of the non-volatile acid.

In U.S. Pat. No. 4,892,944, Mori et al. describe the production of a quat salt by a two-step method involving the reaction of a tertiary amine with a carbonic acid diester to produce a quat carbonate, next, the quat carbonate is mixed with an acid while removing carbon dioxide to product the quat salt of the acid anion.

U.S. Pat. No. 5,438,034 to Walker, describes the preparation of quat ammonium carbonate and quat ammonium bicarbonate by reacting a dialkyldimethylammonium chloride and a metal hydroxide in a C₁-C₄ normal alcohol solvent to form a quat hydroxide. The quat hydroxide is then reacted with carbon dioxide to yield the corresponding quat carbonate and quat bicarbonate. The patent describes the prior art as including the use of an ion exchange resin to convert didecyldimethylammonium bromide to didecyldimethylammonium hydroxide (Talmon et al., Science, 221:1047 (1983)), but states that a large amount of resin was required for conversion of a small amount of quat compound. In fact, the Talmon et al. article describes the process as using a hydroxide ion exchange resin in water at 25° C. in batch mode to convert a quat bromide, which was insoluble in water, to the corresponding quat hydroxide. The article reported that the quat hyroxide was highly soluble in water and spontaneously formed stable vesicles.

Methods for exchanging anions of quat compounds are described in: U.S. Pat. No. 3,523,068 (electrolytic preparation of a quat hydroxide from an aqueous solution of a quat salt of a non-electrolyzable anion, such as sulfate, bisulfate, alkylsulfate, nitrate, carbonate, and bicarbonate); U.S. Pat. No. 4,394,226 (electrolytic preparation of quat hydroxide from quat halides in electrolytic cells separated by a cation exchange membrane); U.S. Pat. No. 4,634,509 (electrolysis of an inorganic acid quat salt in a diaphragm cell to produce the quat hydroxide); U.S. Pat. No. 4,776,929 (production of quat hydroxide by electrolyzing quat bicarbonate salts in a cell having an anode and a cathode compartment separated by a cation exchange membrane); U.S. Pat. No. 5,705,696 (producing a quat ammonium salt having a non-halide anion by contacting an aqueous solution of a quat halide and an alkali metal salt of the desired anion with an organic liquid which is immiscible with the aqueous solution and which is a solvent for the desired quat salt); and U.S. Pat. No. 6,586,632 (conversion of a quat halide to a quat dihydrogen phosphate or bisulfate salt)

As can be seen from the volume of the foregoing work, it would be highly desirable to provide a method to exchange the anion of a quaternary tetraalkylammonium salt in which form the quat is advantageously synthesized, commonly as a halide, with a different anion that provides the quat with desirable properties for particular applications. It would be even more useful if such a method could be operated with a high degree of conversion so that the exchange leaves very low residual content of the first anion with the quat compound. It would be yet more useful if such a method could be operated with a feed concentration of the quat salt that would permit processing at a high rate of throughput.

### SUMMARY OF THE INVENTION

Briefly, therefore the present invention is directed to a novel method of converting a tetraalkylammonium salt of a first anion to a tetraalkylammonium salt of a second anion; the method comprising: forming a solution comprising the tetraalkylammonium salt of the first anion in a solvent which includes water and a polar organic co-solvent; contacting the solution with an ion exchange resin in the form of the second anion and forming a tetraalkylammonium salt of the second anion in solution and an ion exchange resin complexed with the first anion. Optionally, the method can include recovering the solution containing the tetraalkylammonium salt of the second anion.

The present invention is also directed to a novel method of converting a tetraalkylammonium salt of a first anion to a tetraalkylammonium salt of a second anion by contact with an ion exchange resin in the form of the second anion; the method comprising: (a) forming a solution comprising the tetraalkylammonium salt of the first anion in a solvent in which: (i) the tetraalkylammonium salt of the first anion is soluble in an amount of at least about 1% by weight at 25° C., (ii) the tetraalkylammonium salt of the second anion is soluble in an amount of at least about 1% by weight at 25° C., and (iii) the first anion is available for contact with the ion exchange resin; and (b) contacting the solution with the ion exchange resin in the form of the second anion and forming a tetraalkylammonium salt of the second anion in solution and an ion exchange resin complexed with the first anion.

Among the several advantages found to be achieved by the present invention, therefore, may be noted the provision of a method to exchange the anion of a quaternary tetraalkylammonium salt in which form the quat is advantageously synthesized, commonly a halide, with a different anion that provides the quat with desirable. properties for particular applications, and the provision of such a method that could be operated with a high degree of conversion so that the exchange leaves very low residual content of the first anion with the quat compound, and the provision of such a method that could be operated with a feed concentration of the quat salt that would permit processing at a high rate of throughput.

### BRIEF DESCRIPTION OF THE DRAWINGS

FIG. 1 is an illustration of a molecular assembly, here identified as a liposome, that is composed of a tetraalkylammonium salt of a first anion, which is shown here as didecyldimethylammonium bromide salt, in a liquid solution and in contact with an ion exchange resin bead in hydroxide form, where it is seen that the molecular assembly entraps a certain amount of the bromide ions, making them unavailable for exchange with hydroxide ions; and

FIG. 2 shows a graph of the weight of bromide present in successive samples of eluant from the mid-point of a bed of Dowex® Marathon A2, Type 2, strong base ion exchange resin (indicating break through of bromide ions past the sample point) as a function of the concentration of sodium hydroxide in aqueous solution used for regeneration.

### DETAILED DESCRIPTION OF THE PREFERRED EMBODIMENTS

In accordance with the present invention, it has been discovered that a tetraalkylammonium salt of a first anion can be converted to a tetraalkylammonium salt of a second anion by contact with an ion exchange resin in the form of the second anion. The method includes the formation of a solution of the tetraalkylammonium salt of the first anion in a solvent having certain characteristics. It has been found that the solvent is preferably one in which: (i) the tetraalkylammoniun salt of the first anion is soluble in an amount of at least about 1% by weight at 25° C., (ii) the tetraalkylammonium salt of the second anion is soluble in an amount of at least about 1% by weight at 25° C., and (iii) the first anion is available for contact with the ion exchange resin, as will be discussed in detail below. Commonly, the solvent is a mixture comprising water and one or more polar organic co-solvents, such as a lower molecular weight alcohol. Once the solution of the tetraalkylammonium salt of the first anion is formed, that solution is contacted with an ion exchange resin in the form of the second anion and a tetraalkylammonium salt of the second anion is formed in solution and an ion exchange resin complexed with the first anion is also formed. The solution containing the tetraalkylammonium salt of the second anion can then be recovered.

It has been found that the present method can be used at quat concentrations and flow rates that are suitable for commercial industrial applications. For example, quat concentrations of over 10%, or 15%, or even 25% by weight can be processed in the present method. Also, the novel method permits the production of quat products (tetralkylammonium salts of a second anion) having extremely low residual levels of the first anion. When the first anion is bromide, for example, product solutions containing less than 1000 ppm, or even less than about 300 ppm of bromide, or even lower, are obtainable. Even more useful is the fact that the inventors have found that the ion exchange resin can be regenerated frequently and over many cycles without significant loss in exchange efficiency or capacity.

In one step of the present method, a tetraalkylammonium salt of a first anion is placed into solution in a solvent having the aforementioned characteristics, which are described in detail below.

The tetraalkylammonium salt of the first anion can be formed from almost any quaternary tetraalkylammonium compound and almost any anion. However, it has been found to be preferred that the tetralkylammonium compound is one having a nitrogen atom bonded to four (C₁-C₂₄)alkyl groups, or to four (C₁-C₂₀)alkyl groups is more preferred, wherein the nitrogen is left with a net positive charge. As mentioned above, the quat is associated with an anion having a negative charge: Diquat compounds, having two such tetraalkylammonium groups that share a divalent anion, are also included in the present invention. It should be noted that each of the four alkyl groups can be different from any of the other groups, or two or more of the groups can be the same.

It has been found to be preferred that the tetraalkylammonium salt of a first anion comprises a tetra(C₁-C₂₀)alkylammonium salt of a first anion, even more preferred that the tetraalkylammonium salt of a first anion comprises a di(C₁-C₂₀)alkyldimethylammonium salt of a first anion, and even more preferred that the tetraalkylammonium salt of a first anion is a compound that is selected from the group consisting of dioctyldimethylammonium salt, didecyldimethylammonium salt didodecyldimethylammonium salt, ditetradecyldimethylammonium salt, dihexadecyldimethylammonium salt, dioctadecyldimethylammonium salt, and mixtures thereof. In a certain embodiment, the didecyldimethylammonium salt has been found to be particularly useful.

The tetraalkylammonium salts of a first anion that are used in the present method can be synthesized by methods that are well known in the art—such as by the reaction of a trialkylamine and an alkyl halide—or they can be purchased from commercial sources, such as, for example, Albemarle Corporation, Baton Rouge, La.

It should be recognized that while twin-tailed dialkyldimethylammonium compounds are preferred for use in the present invention, the invention also includes the use of double-tailed dialkyldimethylammonium compounds as well. In particular, such compounds as (C₈-C₂₄)alkyl(C₈-C₂₄)alkyldimethylammonium compounds have been found to be useful as the tetraalkylammonium compounds, wherein each of the two alkyl groups can be different.

As described briefly above, a solution is formed that contains an amount of the tetraalkylammonium salt of a first anion in a solvent. Although almost any amount of the tetraalkylammonium salt of a first anion can be placed in the solution up to the solubility limit of the solvent, it has been found to be preferred that the solution comprises at least about 1% tetraalkylammonium salt of the first anion by weight. In order to support more efficient commercial application, it is more preferred that the solution comprises at least about 5% tetraalkylammonium salt of the first anion by weight, at least about 10% tetraalkylammonium salt of the first anion by weight is even more preferred, at least about 15% tetraalkylammonium salt of the first anion by weight is yet more preferred, and at least about 20%, or even more preferably at least about 25% tetraalkylammonium salt of the first anion by weight, or an even higher concentration, is even more preferred. In some embodiments of the present invention, it is preferred that the solution contain the quat salt of the first anion in an amount that is between about 10% and about 30%, by weight, and between about 15% and about 25% is even more preferred.

The solution containing the tetraalkylammonium salt of the first anion can be formed by any method that is effective for such operation. The quat salt can be added to the solvent with stirring until the quat salt goes into solution. If it is helpful, the solvent can be heated somewhat to speed the rate at which the quat salt is solubilized.

In the present invention, the first anion can be any anion that can act as the counterion, or form the anion of a salt, with a quaternary tetraalkylammonium compound. In many cases, it is useful that the first anion be an anion that is commonly used in the synthesis of the quaternary ammonium compound. For example, it is common to react tertiary amines, such as tertiary alkyldimethyl amine, with an alkyl halide, such as alkyl chloride or alkyl bromide, to form the quat alkyl halide salt. However, the first anion of the present invention is not limited to halides. Useful examples of anions that can act as the first anion include such anions as halide, hydroxide, sulfate, bisulfate, sulfonate, phosphate, phosphonate, carbonate, bicarbonate, and mixtures of any of these.

In an embodiment of the present invention, the first anion comprises a halide, and in a preferred embodiment, the first anion can comprise at least one compound that is selected from the group consisting of chloride, bromide, iodide, fluoride, and mixtures thereof. In a more preferred embodiment, the first anion comprises chloride or bromide, and bromide is yet more preferred as the first anion.

In the present method, the first anion is exchanged for a second anion in order to produce a tetraalkylammonium salt of a second anion. While almost any anion can be used as the second anion, it is common to select a second anion that provides desirable properties to the final product quat salt. It is preferred that the second anion is different from the first anion. By this it is meant that if the first anion is bromide, for example, then bromide cannot also act as the second anion.

By way of example, useful anions that can act as the second anion of the present invention include halide, hydroxide, borate, formate, carboxylates, carbonate, bicarbonate, sulfate, bisulfate, sulfite, sulfonate, phosphate, phosphonate, nitrate, chlorate, acetate, and mixtures thereof.

In a preferred embodiment, the second anion is hydroxide. When the first anion is bromide, hydroxide is a particularly useful second anion. In another embodiment, the second anion can be carbonate, or a mixture of carbonate and bicarbonate.

It is preferred that the second anion be one that has a binding coefficient with the ion exchange resin that is about the same as the binding coefficient for the first anion. However, this is not a requirement, and the present method can be successful where the first anion and the second anion have any ratio of binding coefficients with the ion exchange resin.

The solvent that is used in the present invention to dissolve the tetraalkylammonium salt of the first anion is one that must meet certain criteria in order for the method to be successful. The quat salts of the first anion, and quat salts of the second anion are normally amphipathic in nature and exhibit very low true solubility in normal aqueous media. To be practical at a commercial industrial scale of interest, the exchange must be practiced at solute concentrations that are very high when compared with normal solute concentrations in conventional ion exchange operations. In the present case, this means concentrations of the quat salts such as are described below.

In order to be successful, various equilibria that are involved in the present ion exchange process (such as, for example, micellar equilibria, resin-ion to solvent-ion equilibrium, and the like) have to be manipulated such that the ion swap can take place quickly, at high process flow rates, e.g., such as would be required for a successful commercial semi-continuous type of operation. To allow for the mechanics of this overall system to achieve the desired exchanges, the solvent system that is used is of particular importance. The inventors have found that design and selection of the components of the solvent should take into account the following features:
Water, alone, does not provide for adequate mobility and subsequent exchange of anions. This is believed to be due to the molecular assembly structures established in water by the types of amphipathic solutes involved. Water is a good solvent for the inorganic portion of the quat, but it is not a good solvent for the hydrophobic alkyl tails of some substituent groups;
Non-aqueous solvents typically are not capable of providing solvent-separated ion pairs that can participate in the desired ion-exchange at reasonable rates. However, organic solvents provide increased solvation of hydrophobic tails of higher alkyl substituent groups;
Double-tailed surfactants, in particular, exhibit low water compatibility and form viscous “gels” at concentrations ranging from parts-per-million levels on up continuously to fairly elevated concentrations (>50%) in water-only environments; and
Twin-tailed surfactants tend to be special cases within the “double-tail” family. These special examples typically form high viscosity systems in most solvents.

It has been found, however, that solvents can be prepared that meet the various needs of the present method, and permit the desired exchange to proceed quickly and completely. The present solvent is preferably one in which:
(i) the tetraalkylammonium salt of the first anion is soluble in an amount of at least about 1% by weight at 25° C.,
(ii) the tetraalkylammonium salt of the second anion is soluble in an amount of at least about 1% by weight at 25° C., and
(iii) the first anion is available for contact with the ion exchange resin.

Each of these characteristics of the solvent will now be described in detail.

When it is said that a quat salt is soluble in the solvent, it is meant that the described amount of the quat salt can be dissolved in the solvent when the solvent is at a temperature of 25° C. Although it is required that the solvent is one which can contain at least about 1% by weight of the tetraalkylammonium salt of a first anion in solution, and that it is one which can contain at least about 1% by weight of the tetraalkylammonium salt of a second anion in solution, it is preferred that the solvent is one in which the tetraalkylammonium salt of the first anion and the tetraalkylammonium salt of the second anion are soluble in an amount of at least about 5%, by weight, at least about 10%, by weight, is more preferred, at least about 15%, by weight is even more preferred, at least about 20%, by weight, is yet more preferred, and at least about 25%, by weight, or even higher, is even more preferred, where all solubilities are measured at 25° C. It should be understood that when it is said that the solvent should be one in which some amount of the tetraalkylammonium salt of a first anion is soluble and in which some amount of the tetraalkylammonium salt of a second anion is soluble, it is not meant that those amounts of the two quat salts must be soluble in the same volume of solvent simultaneously. It means that the specified amount of each of the two quat salts must be separately soluble in a specified volume of the solvent.

When it is said that the solvent must be one in which the first anion is available for contact with the ion exchange resin, it is meant that the tetraalkylammonium salt of the first anion does not form molecular assemblies that shield the first anion from contact with the ion exchange resin, and also that the tetraalkylammonium salt of the first anion and the ion exchange resin in the form of the second anion are ionized, at least to some degree.

It is known that the tetraalkylammonium salts of a first anion that are of interest in the present method form molecular assemblies in water that shield the first anion from contact with the ion exchange resin. Such molecular assemblies include, but are not limited to, vesicles, micelles, liposomes, liquid crystal arrays, reverse micelles, and the like. Because critical micelle concentration (CMC) values for the quats salt of use in the present invention in water are very low, on the order of 10⁻³ to 10⁻⁹ molar, or even lower, it is almost certain that such molecular assemblies would occur at the concentrations of quat salts that are of interest in the present method if only water is used as the solvent.

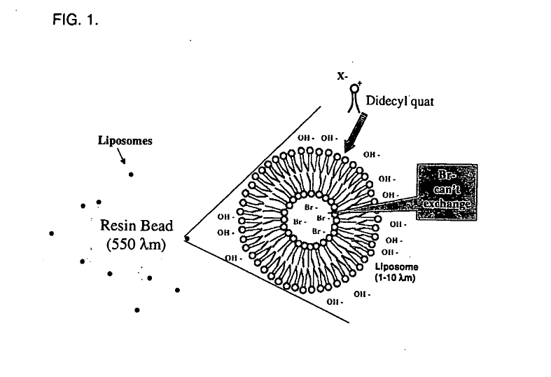

When solvents having high water concentrations are used (low co-colvent: water ratio) it is believed that tetraalkylammonium salts, such as a double-tailed quat, can fold over, shielding some of the first anion from exchange with the resin. This is illustrated in FIG. 1, where a molecular assembly, labeled as a liposome and composed of double-tailed quat salts, didecyldimethylammonium salts in this case, is shown to be shielding an amount of a first anion, which in this case is bromide. Unless and until the molecular assembly is disrupted, allowing access of the trapped bromide ions to the resin bead, ion exchange for the shielded first anions cannot occur.

As mentioned above, it is also preferred that the solvent be one in which the quat salt of a first anion and the ion exchange resin complexed with a second anion are ionized, at least to some degree. It is believed that the presence of the anions in solution promotes exchange rate and degree of completeness.

The availability of the first anion for contact with the ion exchange resin can easily be determined for any particular solvent/quat salt system. One example of a method for this determination is the following test.
1. One mole of a quat salt of a first anion (such as a quat bromide) is dissolved in the solvent to be tested and made up to exactly one liter. The theoretical concentration of the first anion is 1 mole/liter.
2. The indicated, or available, concentration of the first anion in the solution is measured by any appropriate method. For example, when bromide is the first anion, a bromide specific ion probe can be used. The ion specific probe can be calibrated to read moles/liter.
3. The measured concentration of the first anion divided by the theoretical concentration times 100 equals the percent available for exchange.
4. Percent ionization as measured by this test of from about 10% to 100% indicates a solvent in which the first anion is available for contact with the ion exchange resin. It is preferred that a percent ionization of 50%-100% is provided, and a percent ionization of 90% -100% is even more preferred.

This test is most easily used for anions for which an ion specific probe is available (and many are available). An obvious example is the use of a pH probe for sensing hydrogen (and, therefore, hydroxide) concentration.

Another test that is useful for determining whether a solvent is one in which the first anion is available for contact with the ion exchange resin comprises:
1. Prepare a 1 molar solution of quat salt of a first anion in the solvent to be tested as described in the first step of the test described just above.
2. Contact the solution with a stoichiometric excess of an ion exchange resin in the form of the second anion at a temperature that is appropriate for the exchange by preparing a bed of the resin after regeneration to the form of the second anion and flowing the solution through the resin bed. A sample is taken from the first 5% by volume of the solution to exit the resin bed.
3. Measure the total amount of the first anion in the solution. The measuring technique must measure the amount of the first anion in any form.
4. If the concentration of the total amount of the first anion in the solution is less than 10% of the concentration of the first anion in the feed to the column, the solvent is one in which the first anion is available for contact with the ion exchange resin. For example, if the feed solution contains 5%, by weight bromide as bromine (whether present as an ion or in a salt), the solvent is one in which the first anion is available if the concentration of bromine in the solution is less than 0.1×5%, or less than 0.5%. Preferably, the solution will have less than about 5%, and more preferably less than 1% of the concentration of the first anion in the feed to the column.

A preferred solvent for the present invention comprises a mixture of water and one or more polar organic co-solvents. As used herein, an “organic” co-solvent is a compound comprising carbon and hydrogen that is a liquid at room temperature. Also, as used herein, a “polar” co-solvent is a liquid at room temperature that has a dielectric constant that is greater than 4 at 25° C. Preferred polar co-solvents have a dielectric constant that is greater than 6 at 25° C., and more preferred co-solvents have a dielectric constant that is greater than 10 at 25° C.

It is preferred that the co-solvent also be a participating and volatile solvent. A co-solvent is a volatile solvent if it has a boiling point that is lower than the boiling point of pure water under the same conditions, or if it forms a constant boiling azeotrope that has such a boiling point. By way of example, when the tetraalkylammonium salt is a double-tailed molecule, such as a didecyldimethylammonium salt, such co-solvents have been found to facilitate solubilization of the double-tailed molecule in an aqueous environment and allow for good equilibrium and good ion exchange. Examples of suitable organic co-solvents include several classes of compounds, such as:

alcohols, such as methanol, ethanol, isopropyl alcohol, propanol, butanol, isobutyl alcohol, C₁-C₆ alcohols, C₁-C₄ alcohols, and the like, with methanol being a preferred example;

polyalcohols, such as ethylene glycol, propylene glycol, and the like;

esters, such as ethyl acetate, propyl acetate, formates, and the like;

ethers, such as methyl tert-butyl ether, dioxane, glymes, and the like; and

carbonyl-containing solvents, such as acetone, acetaldehyde, and the like.

The polar organic co-solvent must exhibit some compatibility with water. It is preferred that any organic co-solvent that is used is one that is miscible with water.

For the solvents of the present method, it has been found that the ratios of the co-solvent to water are critical to the overall operative efficiency of the present ion exchange operation. Ranges from 10:90 up to 99:1 (co-solvent:water) can be effective under the conditions/controls that are described herein, and depending upon the identity of the tetraalkylammonium compound and upon the first anion and second anion. In general, it is preferred that the ratio of co-solvent: water, by weight, is within the range of from about 50:50 to about 99:1, about 60:40 to about 99:1 is more preferred, about 70:30 to about 98:2 is even more preferred, and about 80:20 to about 95:5 is yet more preferred.

It has generally been found that solvents having a higher ratio of co-solvent to water are preferred for quats having very hydrophobic alkyl substituent groups, e.g., double tailed or twin tailed quats where the alkyl groups are C₁₀-C₂₀, for example, while solvents having a lower ratio of co-solvent to water are preferred for quats having less hydrophobic alkyl substituent groups, e.g., a (C₂-C₆) alkyltrimethylammonium salt.

In one embodiment of the present invention, the solvent comprises a mixture of alcohol and water. It has been found to be preferred that when a mixture of alcohol and water is used as the solvent, the solvent comprises a mixture of a C₁-C₆ alcohol and water in a ratio of from 10:90 to 99:1 by weight. Even more preferred is a solvent that comprises a mixture of a C₁-C₄ alcohol and water in a ratio of from about 50:50 to about 99:1, about 60:40 to about 99:1 is more preferred, about 70:30 to about 98:2 is even more preferred, and about 80:20 to about 95:5 is yet more preferred. An example of a useful solvent for the present invention when the tetraalkylammonium salt is a didecyldimethylammonium salt is a mixture of methanol: water at a ratio of about 85:15, by weight.

It should be understood that when a quat salt is added to a solvent that has two liquid components, the mixture becomes a ternary composition, with at least three major components, water, co-solvent, and quat salt. By way of example, a solution formed by adding 25% by weight of a quat salt to a solvent that comprised an 85:15 by weight mixture of methanol: water, would have a ternary composition, by weight, of 25:64:11, quat salt: methanol: water. When the quat salt is a tetraalkylammonium salt of a first anion, this is one example of a preferred solution for contact with an ion exchange resin in the present invention.

In the present method, the solution containing the quat salt of the first anion is contacted with an ion exchange resin in the form of a second anion. While each and every active site on the resin need not be complexed with the stipulated anion, it is preferred that an ion exchange resin in the form of a second anion, for example, have at least a majority of its active sites complexed to the second anion. It is more preferred that at least about 75%, even more preferred that at least about 85%, and yet more preferred that at least about 90% of the active sites on the resin are complexed with the second anion.

As will be apparent, during the anion exchange step of the present invention, the ion exchange resin will be donating second anions and gaining first anions, so that at any one time, both first and second anions will be present on the resin. When the resin has donated substantially all of the second anions and has substantially all of its active sites complexed with first anions, break through of the first anion will occur and the cycle of exchange will end. Therefore, at the start of an exchange cycle, the ion exchange resin is predominantly in the form of the second anion, and at the end of an exchange cycle the ion exchange resin is predominantly in the form of the first anion. Prior to reuse or recycle, if that is desired, the resin must be regenerated back in the second ion form.

While almost any resin that will exchange anions can be used in the present invention, it is preferred that the resin is a (Type 2) strong base anion exchange resin. The resin can be either a gel-type resin or a macroreticular resin. In some applications, a gel-type resin is preferred, while a macroreticular resin is preferred in other applications. In certain systems, macroreticular resins appear to provide superior exchange. Examples of ion exchange resins that are useful in the present invention include certain Dowex® resins, such as Dowex® Marathon resins, Dowex® Upcore resins, Dowex® Monosphere resins, Dowex® SBR resins, and the like, available from The Dow Chemical Co., Midland, Mich.; SBG, SBM, SBACR resins, and the like, available from Resintech, Inc., West Berlin, N.J.; AG resins, available from Bio-Rad Laboratories, Inc., Hercules, Calif.; Type 2 resins available from QualiChem, Inc., Salem, Va.; Amberlite® IR and IRA resins, available from Rohn & Haas Company, Philadelphia, Pa.; Ionac® resins, available from Sybron Chemicals, at Lanxess Corporation, Pittsburgh, Pa.; and Spectra/Gel® anion exchange resins, available from Spectrum Chromatography, Houston, Tex.; among others.

The ion exchange resin that is used in the present invention should be prepared for use according to the directions provided by the manufacturer. These resins are commonly supplied in chloride form, and if the second anion in the present method is not chloride, then the resins must be converted to the second anion form before they can be used in the method. This is commonly done by contacting the resin in chloride form with a solution containing the second anion of the present process. The solution containing the second anion can be an acid, a salt, or a base, but it must be able to serve as a source for the second anion. For example, if the second anion in the present process is hydroxide, then new resin in chloride form can be contacted with a strong base, such as sodium hydroxide, to convert the chloride form resin into the hydroxide form resin.

The solution containing the tetraalkylammonium salt of the first anion can be contacted with the ion exchange resin in the form of a second anion in any manner that is known in the art. The contact can be in batch mode or in continuous flow mode. When it is said that the contact can be in batch mode, it is meant that an amount of the ion exchange resin in the form of a second ion is intermixed with an amount of the tetraalkylammonium salt of the first anion in the solvent and the mixture is permitted to remain in contact for some period of time—commonly until equilibrium is reached. The resin and solution can then be separated and the quat salt of the second ion can be recovered from the solution.

Commonly, when the present method is used at a commercial scale, it is preferred that the ion exchange resin is placed in a vessel, such as a column, to form a resin bed. The solution containing the tetraalkylammonium salt of a first anion is fed into the vessel containing the bed of the ion exchange resin in the form of the second anion so that the solution passes through the resin bed during which time the first anions exchange with the second anions to form the tetraalkylammonium salt of the second anion and the ion exchange resin complexed with the first anion. Due to selection of the proper solvent, the tetralkylammonium salt of the second anion remains in solution and can be separated from the resin. When the resin bed has become saturated with the first anion, the solution exiting the bed will show an increase in the concentration of the first anion. This point is termed the break through point and can signal the end of the feed step. At this point, the bed can be regenerated, as discussed below, to prepare it for the addition of more feed. The feed and regeneration steps comprises one cycle of bed operation.

When the ion exchange step is performed in a typical ion exchange column, the bed of ion exchange resin is contained in the column and the feed solution is fed to the column at either the top of the bed and removed at the bottom (downflow mode), or fed to the column at the bottom and removed at the top (upflow mode).

When the solution comprising the quat salt of the first anion in a solvent is fed to an ion exchange bed, the feed rate can be expressed in terms of the volume of the feed per unit of time per unit of ion exchange bed surface area, i.e., gallons per minute per square foot of bed surface area (gpm/ft²). This may also be termed the bed loading, or loading rate. In the present invention; the ion exchange step has been found to be successful when the loading rate is between about 0.1 gpm/ft² and about 5 gpm/ft², and a preferred loading rate is between about 0.3 gpm/ft² and about 3.5 gpm/ft², between about 0.6 gpm/ft² and about 2.5 gpm/ft² is even more preferred.

It has been found that a relationship exists between the concentration of the quat salt in the feed and the allowable loading rate, so that as the feed concentration is increased, the maximum allowable loading rate must be decreased in order that the product meet specifications for residual amounts of the first anion. The loading rate and the feed concentration can be adjusted to maximize bed utilization efficiency. It has also been found that the composition of the solvent, as discussed above, is also a factor in arriving at an optimum ion exchange bed operation scheme.

The purpose of the ion exchange step of the present invention is the conversion of the tetraalkylammonium salt of a first anion to the tetraalkylammonium salt of a second anion. Although there is no particular minimum amount of conversion that must occur in order to carry out the present method, it is preferred that the degree of conversion be as high as practical. In an embodiment of the method is it preferred that at least 90 mol % of the tetraalkylammonium salt of the first anion is converted to the tetraalkylammonium salt of the second anion in the contacting step with the ion exchange resin, conversion of at least 99 mol % of the tetraalkylammonium salt of the first anion is more preferred, and conversion of at least 99.9 mol % of the tetraalkylammonium salt of the first anion to the tetraalkylammonium salt of the second anion during the ion exchange contacting step is even more preferred.

Another purpose of the ion exchange step is to reduce the level of the first anion in the product solution to a level that is acceptable for the particular product that is being produced. For example, it is preferred that the solution containing the tetraalkylammonium salt of the second anion that is the product of the ion exchange step (the outflow of an ion exchange column) comprises less than about 3000 ppm of the first anion. When the concentration of the first anion is expressed in terms of parts per million (ppm), what is meant is the parts by weight of the first anion per million parts by weight of final product (usually about 50% by weight of the quat salt and 50% by weight solvent, usually water). When it is said that the particular solution comprises less than a certain amount of the first anion, it is meant that the average concentration of the first anion in the total amount of the solution that has been processed through the column since the last regeneration has less than the stated amount. In other words, product solution exiting a resin bed right after regeneration could have a lower level of the first anion than product exiting the bed near break through. However, the average of the first anion in the total amount of the solution that had passed through the bed could be within the stated amount. In an embodiment of the present invention, it is preferred that the solution containing the tetraalkylammonium salt of the second anion after the ion exchange step comprises less than about 1000 ppm of the first anion, and less than 300 ppm is even more preferred. When the first anion is bromide and the second anion is hydroxide, it is optionally preferred that the solution containing the tetraalkylammonium hydroxide salt after the ion exchange step comprises less than about 300 ppm of bromide reported by weight as chloride.

The present method optionally includes the step of regenerating the ion exchange resin complexed with the first anion and forming ion exchange resin in the form of the second anion. To be of practical commercial utility, the ion exchange resin must be able to be regenerated over repeated cycles. For example, it is preferred that the ion exchange resin is regenerable on a daily, or more frequent, basis, with no significant losses in exchange efficiency over the span of a year of operation.

The ion exchange resin that is useful in the present method can be regenerated by any manner that is known in the art. Generally speaking, the resin is contacted with an excess of a solution that contains the second anion of the present invention. For example, when the first anion comprises a halide, the regeneration of the ion exchange resin in halide form comprises contacting it with a solution that contains the second anion to form ion exchange resin in the form of the second anion. If the second anion is hydroxide, then the ion exchange resin can be regenerated by contacting it with an excess of a strong hydroxide base, such as sodium hydroxide. Although the regenerating compound (acid, base, or salt) can be in solution in any liquid, it is typical to regenerate the ion exchange resin with an aqueous solution of the compound. The sodium bromide salt solution that is a by-product of regeneration of a resin in bromide form can be sent to a recovery process, if desired, where sodium hydroxide and bromine can be recovered.

When sodium hydroxide is used to regenerate an ion exchange resin, it has been found to be preferred to contact the resin with a molar excess of the sodium hydroxide relative to the moles of the first anion that are complexed with the resin at the time regeneration is begun. For example, if the resin is complexed with 1 mole of a first anion, such as a halide, then it is preferred that from about 2 to about 10 moles of sodium hydroxide are contacted with the resin in order to successfully regenerate the resin into hydroxide form. It is more preferable that from about 3 to about 5 moles caustic per mole first anion on the resin be used, and even more preferred. that the molar ratio of caustic: first anion is between about 4 and 4.5.

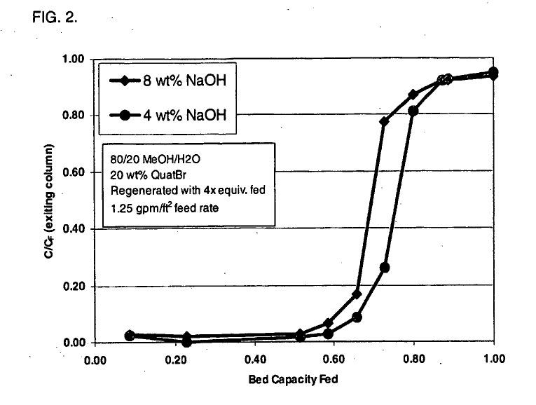

The inventors have found that the bed regeneration procedures for the ion exchange resin system have a significant effect on the economics of the method of the invention. Factors such as the concentration of the base in the regenerating solution, the flow rate of the regenerating solution, and the total amount of base that is used relative to the amount of first anion that must be displaced from the resin, have all been found to be useful for control of the efficiency of the regeneration process. For example, FIG. 2 shows a graph of the bromide concentration profile taken at the mid-point of an ion exchange bed. A 20% quat bromide stream was fed to this bed and converted to hydroxide form. The bed was then regenerated with 4 equivalents of sodium hydroxide per bromide equivalent fed. Runs using 4% and then 8% sodium hydroxide for regeneration were completed. The breakthrough curve following the regenerations with 4% sodium hydroxide was sharper than the curve which follows regeneration with 8% sodium hydroxide, and occurs later during the feed cycle. This indicates regeneration with more dilute caustic can increase effective bed capacity, or could be used to reduce the amount of caustic required for regeneration at an equivalent capacity demonstrated with 8% regenerations.

When the first anion is a halide and the second anion is hydroxide, it is preferred that the regeneration of the ion exchange resin in halide form comprises contacting the resin in an ion exchange resin bed with an aqueous solution comprising at least about 1%-25% sodium hydroxide by weight at a rate of at least about 0.1 gallons per minute per square foot of ion exchange bed surface area (gpm/ft²). It has been found that dilute solutions of sodium hydroxide make the most effective use of hydroxide equivalents, but the volumes of dilute streams often result in higher operating costs. As an economic balance, hydroxide solution bed at a rate between about 0.5 and 2.5 gpm/ft², and even more preferred that the aqueous solution of sodium hydroxide is contacted with the ion exchange bed at a rate between about 0.6 and 1.5 gpm/ft².

Although the feed step and the regeneration of the ion exchange bed can proceed in either upflow or downflow mode, when the bed is in a vessel or column it has been found to be preferred that the regenerating solution is contacted with the ion exchange. bed in a mode that is opposite to that used for the feed step. By way of example, when the bed is fed in upflow mode, then regeneration in downflow mode is preferred, and vice versa.

After regeneration of the ion exchange resin, the present method can optionally include recycling all or a part of the regenerated ion exchange resin in the form of the second anion for use in contacting the solution containing the tetraalkylammonium salt of a first anion. Of course, this can recur a number of times.

It should be noted that the ion exchange resin can be rinsed or purged with a liquid, such as water, between any of the steps of the present method.

In the case where the second anion is something other than hydroxide, for example, when it is carbonate, phosphate, sulfate, or sulfonate, or the like, it may be preferred to regenerate the spent ion exchange resin in two steps, rather than in one step. For example, if a quat bromide is converted to a quat carbonate in an ion exchange step, then the spent resin ultimately must be regenerated from a bromide form to a carbonate form. This can be done by a first regeneration step that includes contacting the resin with a strong base hydroxide, as discussed above, to convert the resin to the hydroxide form. This step is followed by contacting the resin with a solution containing carbonate or bicarbonate anions in order to convert the resin back to the carbonate form. In this case, it may be necessary to use a significant excess of the carbonate solution to convert the resin back to the desired starting form.

In a particularly useful embodiment of the present invention, the tetraalkylammonium salt of a first anion is a tetraalkylammonium bromide that is produced by the reaction of a trialkylamine with an alkyl bromide. Trialkylamines that are useful in the method include (C₁-C₂₀)alkyldimethylamines, and useful alkyl bromides include (C₁-C₂₀)alkyl bromides. This embodiment is illustrated in the general scheme shown below: 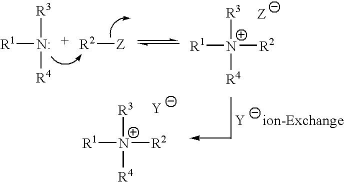
where:
R¹, R², R³, and R⁴ are each separately an alkyl;
Z⁻ is a halide; preferably a bromide;
Y⁻ is a second ion; as described above.

In a preferred embodiment, the trialkylamine is decyldimethylamine, the alkyl bromide is decyl bromide, and the tetraalkylammonium salt is didecyldimethylammonium bromide.

This method can be used for the direct production of a tetraalkylammonium carbonate and/or bicarbonate salt when the second anion is carbonate or a mixture of carbonate and bicarbonate. It has been found that this method is particularly useful when the first anion comprises bromide and wherein the method further comprises regeneration of the ion exchange resin by contacting the ion exchange resin in bromide form with sodium hydroxide to convert at least a portion of the ion exchange resin to anion is carbonate or a mixture of carbonate and bicarbonate. It has been found that this method is particularly useful when the first anion comprises bromide and wherein the method further comprises regeneration of the ion exchange resin by contacting the ion exchange resin in bromide form with sodium hydroxide to convert at least a portion of the ion exchange resin to the hydroxide form and to form sodium bromide; and contacting the ion exchange resin in hydroxide form with a source of carbonate anions or a mixture of carbonate and bicarbonate anions and converting at least a portion of the ion exchange resin in hydroxide form to the carbonate form or a mixture the carbonate form and the bicarbonate form.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### General Procedures

Production of Dialkyldimethylammonium Bromide:

A 1-liter, creased, 4-necked round bottom Pyrex flask equipped with mechanical stirrer, 250-milliliter (ml) addition funnel, temperature probe, heating mantle, and water-cooled total reflux condenser was used. The flask was initially charged with 200 grams (1.08 mole) of ADMA-10 (decyldimethylamine, available from Albemarle Corporation, Baton Rouge, La.), and 239 grams (1.08 mole) decyl bromide was placed in the addition funnel. The stirrer was turned on and the reactor was heated to 65° C. The decyl bromide was added dropwise to the ADMA-10 as the temperature of the reactor was allowed to rise from 65° C. to 142° C. The addition funnel was then charged with 110 grams of methanol, and the methanol was added dropwise to the solution as the temperature of the reactor was allowed to fall to 90° C. After the entire volume of the methanol was added to the reaction mixture, the heat and stirring were ceased and the intermediate quaternary ammonium (quat-Br) solution was allow to cool.

### EXAMPLES 1-11

These examples illustrate ion-exchange procedures used to exchange the halide anions, such as bromide, of a quaternary ammonium compound with another anion, such as bicarbonate.

A 28 mm×150 mm glass chromatography column was set up with a cotton plug in the bottom. The glass column was charged with 40 grams of Amberlite A-27 Strong Base/Macroreticular Type Ion Exchange Resin (available from Dow Chemical Company), and washed with 50 ml distilled water, pH 7. The column was charged with HCO₃⁻ ions by eluting a solution of 30 grams of NaHCO₃ in 400 ml distilled water through the column.

Five grams of quat-Br, such as that made by the process described above, was dissolved in 5 grams of methanol. This quat bromide solution was placed on the resin bed and eluted with a 2:1 mixture of methanol:water and collected in the following aliquots.

TABLE 1
One-Step Column Charging - Analysis of the Final
Quat Product Eluted From an Ion-Exchange Column

| Example/Sample | | | |
|---|---|---|---|
| No. | Volume | pH | Visual Description |
| 1 | 0 ml | 8.5 | colorless |
| 2 | 25 ml | 8.0 | colorless |
| 3 | 10 ml | 7.5-8.0 | colorless |
| 4 | 10 ml | | pale yellow |
| 5 | 10 ml | 7.5-8.0 | pale yellow |
| 6 | 10 ml | | pale yellow |
| 7 | 10 ml | | pale yellow/cloudy |
| 8 | 10 ml | | cloudy |
| 9 | 10 ml | | cloudy |
| 10 | 10 ml | | very pale |
| | | | yellow/cloudy |
| 11 | 10 ml | 7.0 | clear |

Samples 4, 6, and 8 were selected for analysis by ion chromatography to determine the presence of residual chloride ions that were present on the resin prior to use and the presence of HCO₃⁻ ions.

TABLE 2
One-Step Ion Chromatography Analysis.

| Example/Sample | | | |
|---|---|---|---|
| No. | Cl⁻ | Br⁻ | HCO₃⁻ |
| 4 | 724 ppm | — | Yes |
| 6 | 1011 ppm | — | yes |
| 8 | 212 ppm | — | yes |

### EXAMPLES 12-20

These examples describe a two-step regeneration protocol for a previously used ion exchange column, such as the column resulting from the procedure described in Examples 1-11, to remove the bromide ions present on the resin that were extracted from the intermediate quat bromide, and the subsequent use of the regenerated column for ion exchange.

The previously used resin, such as that described in Examples 1-11, was washed with 200 ml distilled water to purge the methanol. The column was then charged with OH⁻ ions by eluting 250 ml of 10% NaOH in distilled water through the ion exchange bed and then washed with distilled water. The resin was then charged with HCO₃⁻ ions by eluting a solution of 17.47 grams of NaHCO₃ dissolved in 300 ml of distilled water through the ion exchange bed. The column was then washed with 80 ml of distilled water, followed by washing with 95% methanol/5% water.

Then 14.52 grams of the quat-Br solution prepared as described in the General Procedures 1 was dissolved in 5.82 grams methanol, and introduced to the charged column. Elution was carried out with a mixture of 95% methanol/5% water, and collected in the following 15 ml aliquots detailed in Table 3.

TABLE 3
Two-Step Column Charging - Analysis of the Final
Quat Product Eluted From an Ion-Exchange Column

| Example/Sample No. | Visual Description |
|---|---|
| 12 | discard |
| 13 | colorless |
| 14 | colorless |
| 15 | light yellow/cloudy |
| 16 | yellow/clear |
| 17 | yellow/clear |
| 18 | light yellow |
| 19 | very slight yellow |
| 20 | colorless |

Samples 16 and 17 were chosen for analysis by ion chromatography to determine the presence of residual bromide ions and the presence of HCO₃⁻ ions.

TABLE 4
Two-step ion chromatography analysis.

| Example/Sample No. | Br— | HCO₃— |
|---|---|---|
| 17 | 669 ppm | 2.53% |
| 18 | 1213 ppm | 2.65% |

### EXAMPLES 21-23

These examples illustrate exchange of the bromide anion of a quaternary tetraalkylammonium bromide salt with a second anion in a 2-inch diameter column, and regeneration of the column.

Column Preparation

Two columns, each 2″×48″, were loaded with 1500 g of Dowex Marathon A2 resin which represents approximately 2.69 exchange equivalents/bed. This resin as supplied by Dow Chemical Co., Midland, Mich., is in the chloride form. Each was flushed with several liters of water and then put into the desired hydroxide or bicarbonate form as follows.

Column Regeneration

A typical regeneration to the hydroxide form involves running enough water through the column to remove any remaining organics from the previous run, typically several liters of water. This is followed by six or more liters of 8 wt % caustic, and then additional water until the effluent is neutral. Since countercurrent regeneration was difficult in the laboratory as equipped, the columns were inverted resulting in what is essentially countercurrent regeneration. After inversion, what was formerly the top of the column which had seen the most caustic and should be the most halide free, became the bottom of the column. The column was ready for use after inversion.

In some cases, recycle caustic was used in the regeneration. In that case, the later fractions of caustic used in regeneration contain little bromide and thus were used for the first caustic fractions of the next column regeneration. This was always followed by several fractions of fresh caustic.

When the bicarbonate form was desired, a two-step regeneration was employed. The column was first put into the hydroxide form as above, and then two equivalents of sodium bicarbonate in water (approximately 8 wt %) were passed through the column. Then additional water was passed until the effluent was neutral. The column was then inverted and was ready for use.

Typical Anion Exchange Procedure

After the column is regenerated and inverted, the void volume water (approximately 50% of the column volume) has to be replaced with the solvent to be used in the exchange. Approximately one liter, more or less, of the desired solvent is passed through the column. The feed is then added and the flow rate adjusted to approximately 20 ml/min. Aliquots, generally 500 ml each at this scale, are collected and analyzed and/or combined. After all of the feed is on the column, an additional one to two liters of solvent is passed through the column to completely flush all of the quat from the column. The column is then ready for a water flush and then inversion and regeneration. All bromide values reported in Part 2 were determined by classical silver nitrate titration.

### EXAMPLE 21

### Conversion of Quat Bromide to Quat Bicarbonate

One column was put into the bicarbonate form and used for this experiment.

The feed for this run was 1365 g of 80% didecyldimethylammonium bromide in methanol, 2457 g additional methanol, and 1638 g water. This resulted in a quat bromide:methanol:water ratio of 20:50:30. Initially 700 ml of methanol/water (ratio 62.5:37.5 by volume) was passed through the column to displace the water and condition the column to the correct methanol/water ratio. The feed mixture was put onto the column and was followed by a liter of methanol/water. Aliquots were collected and analyzed as shown in Table 5.

TABLE 5
Residual bromide in effluent of ion exchange column
as a function of volume of feed to the column.

| | Elapsed | Volume | Flow rate | Bromide | |
|---|---|---|---|---|---|
| Fraction | time (min) | (ml) | (ml/min) | (ppm) | Comments |
| 0 | 22 | 1050 | — | | |
| 1 | 26 | 500 | 22.73 | | |
| 2 | 26 | 500 | 19.23 | | |
| 3 | 26 | 500 | 19.23 | | |
| 4 | 26 | 500 | 19.23 | 151 | Frac. |
| | | | | | 3 & 4 |
| 5 | 26 | 500 | 19.23 | | |
| 6 | 27 | 500 | 18.52 | | |
| 7 | 27 | 500 | 18.52 | | |
| 8 | 26 | 500 | 19.23 | 135 | Frac. |
| | | | | | 7 & 8 |
| 9 | 26 | 510 | 19.62 | | |
| 10 | | 550 | — | | |
| 11 | | 480 | — | | |
| 12 | 30 | 500 | 16.67 | 105 | Frac. |
| | | | | | 11 & 12 |
| 13 | 15 | 250 | 16.67 | | |
| 14 | 10 | 250 | 25 | 76 | Frac. |
| | | | | | 13 & 14 |

Notes:
Feed was 20% quat bromide, 50% methanol, 30% water.

### EXAMPLE 22

### Conversion of Quat Bromide to Quat Hydroxide with Polish

Two columns were regenerated into the hydroxide form by the standard procedure outlined above.

The feed for this run was 1024 g of 80% didecyldimethylammonium bromide in methanol, 1843 g additional methanol, and 1228 g water. This resulted in a quat bromide:methanol:water ratio of 20:50:30. Initially 700 ml of methanol/water (ratio 62.5:37.5 by volume) was passed through the column to displace the water and condition the column to the correct methanol/water ratio. The feed mixture was put onto the column and was followed by methanol/water. Aliquots were collected and analyzed as shown in the Tables 6 and 7, which follow.

TABLE 6
Loading rate for first of two ion exchange columns.

| | Elapsed | Volume | Flow rate | Bromide | |
|---|---|---|---|---|---|
| Fraction | time (min) | (ml) | (ml/min) | (ppm) | Comments |
| 0 | 75 | 970 | 12.93 | | |
| 1 | 44 | 500 | 11.36 | | |
| 2 | 35 | 500 | 14.29 | | |
| 3 | 35 | 500 | 14.29 | | |
| 4 | 35 | 500 | 14.29 | | |
| 5 | 34 | 500 | 14.71 | | |
| 6 | 40 | 500 | 12.50 | | |
| 7 | 36 | 500 | 13.89 | | |
| 8 | 42 | 505 | 12.02 | | |
| 9 | 39 | 500 | 12.82 | | |
| 10 | 39 | 500 | 12.82 | | |
| 11 | 34 | 340 | 10 | | |
| 12 | 30 | 500 | 16.67 | 105 | |

Notes:
Feed was 20% quat bromide, 50% methanol, 30% water. 75% column equivalent charge.

The effluent from the first column was fed to a second column, as a “polish” column, and the output from that column analyzed for residual bromide.

TABLE 7
Residual bromide in effluent of second ion exchange column
as a function of volume of feed to the column.

| | Elapsed | Volume | Flow rate | Bromide | |
|---|---|---|---|---|---|
| Fraction | time (min) | (ml) | (ml/min) | (ppm) | Comments |
| 0 | 45 | 1020 | 22.67 | | |
| 1 | 35 | 500 | 14.29 | | |
| 2 | 30 | 500 | 16.67 | | |
| 3 | 30 | 500 | 16.67 | | |
| 4 | 29 | 500 | 17.24 | 164 | |
| 5 | 27 | 500 | 18.52 | | |
| 6 | 28 | 500 | 17.86 | 160 | |
| 7 | 31 | 560 | 18.06 | | |
| 8 | 26 | 440 | 16.92 | 158 | |
| 9 | 30 | 560 | 18.67 | | |
| 10 | 28 | 620 | 22.14 | 188 | |

Notes:
Feed was 20% quat bromide, 50% methanol, 30% water. Bromide content in combined fractions 1-11 was 169 ppm Br.

### EXAMPLE 23

### Conversion of Quat Bromide to Quat Hydroxide at Higher Concentration

The feed for this run was 1279.7 g of 80% didecyldimethylammonium bromide in methanol, 1843 g additional methanol, and 1228 g water. This resulted in a quat bromide:methanol:water ratio of 23.5:48.25:28.25. The feed quat bromide had been prepared with 1% excess amine in an effort to minimize unreacted decylbromide. The bromide concentration in the effluent from the ion exchange column is shown in Table 8.

TABLE 8
Residual bromide in effluent of ion exchange column
as a function of volume of feed to the column.

| | Elapsed | Volume | Flow rate | Bromide | |
|---|---|---|---|---|---|
| Fraction | time (min) | (ml) | (ml/min) | (ppm) | Comments |
| 0 | 66 | 1030 | 15.61 | | |
| 1 | 43 | 530 | 12.33 | 223 | |
| 2 | 40 | 500 | 12.50 | 492 | |
| 3 | 40 | 560 | 14.0 | 473 | |
| 4 | 34 | 450 | 13.24 | 246 | |
| 5 | 35 | 510 | 14.57 | 267 | |
| 6 | 36 | 570 | 15.83 | 276 | |
| 7 | 32 | 500 | 15.63 | 308 | |
| 8 | 35 | 500 | 14.29 | 327 | |
| 9 | 35 | 500 | 14.29 | 7003 | |
| 10 | 30 | 490 | 16.33 | 14625 | |
| 11 | 14 | 220 | 15.71 | 3968 | Cloudy |

Notes:
Feed was 23.5% quat bromide, 48% methanol, 28.25% water.

This run clearly shows that low bromide can be obtained in a single pass through the column. Also, it clearly shows bromide breakthrough toward the end of the run. The reduced amount of bromide in fraction 11 is the result of dilution with additional methanol/water used to flush any remaining quat from the column at the end of the run.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions by those of ordinary skill in the art without departing from the scope of the invention, it is intended that all matter contained in the above description and shown in the accompanying drawings shall be interpreted as illustrative and not in a limiting sense. In addition it should be understood that aspects of the various embodiments may be interchanged both in whole or in part.

What is claimed is:

1. A method of converting a tetraalkylammonium salt of a first anion to a tetraalkylammonium salt of a second anion; the method comprising:
forming a solution comprising the tetraalkylammonium salt of the first anion in a solvent which includes water and a polar organic co-solvent;
contacting the solution with an ion exchange resin in the form of the second anion and forming a tetraalkylammonium salt of the second anion in solution and an ion exchange resin complexed with the first anion.

2. The method according to claim 1, further comprising recovering the solution containing the tetraalkylammonium salt of the second anion.

3. The method according to claim 1, wherein the solution comprising the tetraalkylammonium salt of the first anion comprises at least about 5% tetraalkylammonium salt of the first anion by weight.

4. The method according to claim 1, wherein the solution comprising the tetraalkylammonium salt of the first anion comprises at least about 10% tetraalkylammonium salt of the first anion by weight.

5. The method according to claim 1, wherein the solution comprising the tetraalkylammonium salt of the first anion comprises at least about 20% tetraalkylammonium salt of the first anion by weight.

6. The method according to claim 1, wherein the solvent which includes water and a polar organic co-solvent is one in which the tetraalkylammonium salt of the first anion and the tetraalkylammonium salt of the second anion are each soluble in an amount of at least about 5% by weight.

7. The method according to claim 1, wherein the solvent which includes water and a polar organic co-solvent is one in which the tetraalkylammonium salt of the first anion and the tetraalkylammonium salt of the second anion are each soluble in an amount of at least about 20% by weight.

8. The method according to claim 1, wherein the first anion comprises at least one anion that is selected from the group consisting of halide, hydroxide, sulfate, bisulfate, sulfonate, phosphate, phosphonate, carbonate, bicarbonate, and mixtures thereof.

9. The method according to claim 1, wherein the first anion comprises at least one anion that is selected from the group consisting of chloride, bromide, iodide, fluoride, and mixtures thereof.

10. The method according to claim 1, wherein the first anion comprise chloride, or bromide, or a mixture thereof.

11. The method according to claim 1, wherein the first anion comprises bromide.

12. The method according to claim 1, wherein the second anion is different from the first anion and comprises at least one anion that is selected from the group consisting of halide, hydroxide, borate, formate, carboxylates, carbonate, bicarbonate, sulfate, bisulfate, sulfite, sulfonate, phosphate, phosphonate, nitrate, chlorate, acetate, and mixtures thereof.

13. The method according to claim 11, wherein the second anion comprises hydroxide.

14. The method according to claim 11, wherein the second anion is carbonate or a mixture of carbonate and bicarbonate.

15. The method according to claim 1, wherein the solvent comprises a mixture of water and a co-solvent comprising at least one polar organic liquid that is selected from the group consisting of alcohols, polyalcohols, esters, ethers, and carbonyl-containing solvents.

16. The method according to claim 1, wherein the solvent comprises a mixture of water and a co-solvent comprising at least one polar organic liquid that is selected from the group consisting of methanol, ethanol, isopropyl alcohol, propanol, butanol, isobutyl alcohol, C₁-C₆ alcohols, C₁-C₄ alcohols, ethylene glycol, propylene glycol, ethyl acetate, propyl acetate, formates, methyl tert-butyl ether, dioxane, glymes, acetone, and acetaldehyde.

17. The method according to claim 1, wherein the solvent comprises a mixture of alcohol and water.

18. The method according to claim 1, wherein the solvent comprises a mixture of a C₁-C₄ alcohol and water in a ratio of alcohol:water of from about 50:50 to 99:1 by weight.

19. The method according to claim 1, wherein the solvent comprises a mixture of methanol and water in a ratio of methanol:water from 70:30 to 99:1 by weight.

20. The method according to claim 1, wherein step of contacting the solution with an ion exchange resin comprises flowing the solution containing the tetraalkylammonium salt of a first anion into a vessel containing a bed of the ion exchange resin in the form of the second anion so that the solution passes through the resin bed causing the first anions to exchange with the second anions to form the tetraalkylammonium salt of the second anion and the ion exchange resin complexed with the first anion.

21. The method according to claim 1, wherein the tetraalkylammonium salt of a first anion comprises a tetra(C₁-C₂₀)alkylammonium salt of a first anion.

22. The method according to claim 1, wherein the tetraalkylammonium salt of a first anion comprises a di(C₁-C₂₀)alkyldimethylammonium salt of a first anion.

23. The method according to claim 1, wherein the tetraalkylammonium salt of a first anion is a compound that is selected from the group consisting of dioctyldimethylammonium halide salt, didecyldimethylammonium halide salt didodecyldimethylammonium halide salt, ditetradecyldimethylammonium halide salt, dihexadecyldimethylammonium halide salt, dioctadecyldimethylammonium halide salt, and mixtures thereof.

24. The method according to claim 1, wherein the ion exchange resin is a strong base anion exchange resin.

25. The method according to claim 24, wherein the ion exchange resin is a gel type resin or a macroreticular resin.

26. The method according to claim 1, wherein at least 90 mol % of the tetraalkylammonium salt of the first anion is converted to the tetraalkylammonium salt of the second anion.

27. The method according to claim 1, wherein at least 99 mol % of the tetraalkylammonium salt of the first anion is converted to the tetraalkylammonium salt of the second anion.

28. The method according to claim 1, wherein at least 99.9 mol % of the tetraalkylammonium salt of the first anion is converted to the tetraalkylammonium salt of the second anion.

29. The method according to claim 2, wherein the solution containing the tetraalkylammonium salt of the second anion comprises less than about 3000 ppm of the first anion.

30. The method according to claim 2, wherein the solution containing the tetraalkylammonium salt of the second anion comprises less than about 1000 ppm of the first anion.

31. The method according to claim 2, wherein the solution containing the tetraalkylammonium salt of the second anion comprises less than about 300 ppm of the first anion.

32. The method according to claim 13, wherein the solution containing the tetraalkylammonium salt of hydroxide comprises less than about 300 ppm of bromide reported by weight as chloride.

33. The method according to claim 1, further comprising:
regenerating the ion exchange resin in the form of the first anion and forming ion exchange resin in the form of the second anion.

34. The method according to claim 33, wherein the first anion comprises a halide and the regeneration of the ion exchange resin in halide form comprises contacting it with a base having as its anion the second anion and forming ion exchange resin in the form of the second anion.

35. The method according to claim 33, further comprising recycling all or a part of the regenerated ion exchange resin in the form of the second anion for use in the step comprising contacting the solution with an ion exchange resin in the form of the second anion and forming a tetraalkylammonium salt of the second anion.

36. The method according to claim 34, wherein the ion exchange resin is contained in a vessel in a bed, and the method comprises contacting the resin in the ion exchange resin bed with an aqueous solution comprising sodium hydroxide in a concentration of from about 1%-25% by weight at a rate of at least about 0.1 gallons per minute per square foot of ion exchange bed surface area (gpm/ft²).

37. The method according to claim 36, wherein the aqueous solution comprises sodium hydroxide in a concentration of from about 2%-12% by weight.

38. The method according to claim 36, wherein the aqueous solution comprises sodium hydroxide in a concentration of from about 4%-8% by weight.

39. The method according to claim 36, wherein the aqueous solution of sodium hydroxide is contacted with the ion exchange bed at a rate of at least about 0.3 gpm/ft².

40. The method according to claim 36, wherein the aqueous solution of sodium hydroxide is contacted with the ion exchange bed at a rate of at least about 1 gpm/ft².

41. The method according to claim 36, wherein the aqueous solution of sodium hydroxide is contacted with the ion exchange bed at a rate of at least about 2 gpm/ft².

42. The method according to claim 36, wherein the aqueous solution of sodium hydroxide is contacted with the ion exchange bed in a flow mode that is opposite the flow mode that was used while the solution containing tetraalkylammonium salt of the first anion was fed to the bed.

43. The method according to claim 1, wherein the tetraalkylammonium salt of a first anion is a tetraalkylammonium bromide that is produced by the reaction of a trialkylamine with an alkyl bromide.

44. The method according to claim 43, wherein the trialkylamine is a (C₁-C₂₀)alkyldimethylamine and the alkyl bromide is a (C₁-C₂₀)alkyl bromide.

45. The method according to claim 43, wherein the trialkylamine is decyldimethylamine, the alkyl bromide is decyl bromide, and the tetraalkylammonium salt is didecyldimethylammonium bromide.

46. The method according to claim 44, wherein the second anion is carbonate or a mixture of carbonate and bicarbonate.

47. The method according to claim 46, wherein the first anion comprises bromide and wherein the method further comprises regeneration of the ion exchange resin by;
contacting the ion exchange resin in bromide form with sodium hydroxide to convert at least a portion of the ion exchange resin to the hydroxide form and to form sodium bromide; and contacting the ion exchange resin in hydroxide form with a source of carbonate anions or a mixture of carbonate and bicarbonate anions and converting at least a portion of the ion exchange resin in hydroxide form to the carbonate form or a mixture the carbonate form and the bicarbonate form.

48. A method of converting a tetraalkylammonium salt of a first anion to a tetraalkylammonium salt of a second anion by contact with an ion exchange resin in the form of the second anion; the method comprising:
(a) forming a solution comprising the tetraalkylammonium salt of the first anion in a solvent in which:
(i) the tetraalkylammonium salt of the first anion is soluble in an amount of at least about 1% by weight at 25° C.,
(ii) the tetraalkylammonium salt of the second anion is soluble in an amount of at least about 1% by weight at 25° C., and
(iii) ) the first anion is available for contact with the ion exchange resin; and
(b) contacting the solution with the ion exchange resin in the form of the second anion and forming a tetraalkylammonium salt of the second anion in solution and an ion exchange resin complexed with the first anion.
